(19) United States
(12) Patent Application Publication
(10) Pub. No.: US 2005/0221995 A1
(43) Pub. Date: Oct. 6, 2005

(54) FULLERENE LUBRICANT

(76) Inventor: Harold M. Lowe, Crossville, TN (US)

Correspondence Address: GREENBERG & LIEBERMAN, 314 PHILADELPHIA AVE., TAKOMA PARK, MD, 20912, US

(21) Appl. No.: 10/708,937

(22) Filed: Apr. 1, 2004

(51) Int. Cl. A61F 2/28; C10M 125/02

(52) U.S. Cl. 508/113; 623/22.13; 128/898

(57) ABSTRACT

Fullerenes are employed to provided lubrication at an interface. In the human body, fullerenes are introduced into the joint space to make up for loss of synovial fluid. In non human applications, the fullerenes are introduced into the interface to assist in lubrication, much like motor oil. Inert and not soluble in water, fullerenes act like tiny soccer balls rolling over one another to provide the desired amount of lubrication.

### BACKGROUND OF INVENTION

The present invention relates to lubrication of joints. More particularly, the present invention employs fullerenes, with their unique atomic geometry, to ease mechanical movements in living bodies, as well as in other mechanical nonhuman instances.

The human body is a great and magnificent creation. One of the requisite parts of the human body is the skeletal system, allowing interior reliable structural integrity for the human body while permitting some movement. Where bones of the skeletal system meet is called a “joint.” Joints are important because they allow bones to communicate with one another so that the human body is structurally sound, but can bend and flex when necessary.

There are three general types of joints, and each represents a different medium for interfacing between bones. Fibrous joints involve the use of fibrous connective tissue between bones; cartilaginous joints involve the use of cartilage between bones; synovial joints involve the use of thick fluid lubricating between bones. When joints become painful, it is often because of the loss or lack of fluid lubrication.

Various life impacting situations occur because of joint lubrication failure. Some individuals can no longer walk normally and must resort to canes or wheelchairs. Other individuals face daily pain discomfort, and can only mitigate the discomfort by taxing their kidneys with pain relievers. There is a need for a compound and treatment to lubricate joints that have lost their natural lubrication. Of course, because the user of such a compound and treatment would be a human, the compound and treatment must be completely safe.

There are a variety of compounds sold to replace fluid loss in a joint, as opposed to drugs to relieve joint pain; however, none of them is completely safe for all users. There is a need for an inert compound and treatment for lubrication of human joints that does not impact the human body but for lubricating joints.

One compound sold to replace fluid loss in a joint is Hylan G-F 20 which is an FDA-approved therapy that provides lubrication for a user″s knees. Its details are available at http://us.synvisc.com/index_flash.asp. It also acts as a “shock absorber” to cushion the user″s knee joint. As described by its manufacturer, Hylan G-F 20 is an elastic and viscous fluid that is made from a substance called hyaluronan that is found in normal joint fluid. Hyaluronan acts as a shock absorber and lubricant in a typical human joint and is needed for a typical human joint to work properly. The manufacturer cites that Hylan G-F 20 is indicated for the treatment of pain in osteoarthritis of the knee in patients who have failed to respond adequately to conservative nonpharmacologic therapy, and simple analgesics, e.g., acetaminophen. Hylan G-F 20 is injected directly into the knee joint and is not a pharmaceutical. Unfortunately, the manufacturer notes that a user should advise its doctor if it is allergic to products from birds, such as feathers, eggs, or poultry. Unlike the present invention, Hylan G-F 20 is not a simple compound that is uniformly well tolerated throughout the human population.

Another compound sold to provide lubrication for joints is glucosamine. A product containing glucosamine can be found at http://www.bradventures.com/nutrition/products/performanceenhancers/jointmatrix.shtml. The product, sold under the trademark “Joint Matrix” touts itself as containing the same basic molecule that comprises human connective tissue. Glucosamine, it is advanced, helps the user to make new proteoglycans, substances that enable the user″s worn connective tissue to become smooth and resilient. Moreover, it is asserted that glucosamine also comprises 50 percent synovial fluid, the lubricating component found in some of the user″s joints. Unlike the present invention, glucosamine increases insulin resistance, and so it is not terribly safe for pregnant women, as well as diabetics.

Moreover, products like Joint Matrix™ typically contain, as does Joint Matrix™, organic substances to enhance the user″s own bodily anti-inflammatory ability. Unlike the present invention, organic substances that affect the human body do much more than is necessary to replace joint fluid loss. Unlike the present invention, organic substances are not inert.

Thus, there is a need for a compound and treatment that lubricates joints in the human body without having any possibility of ill effects. Further still, there is a need for a compound and treatment that lubricates joints in the human body that is readily available and inexpensive.

In addition, typical nonhuman mechanical interfaces are conventionally lubricated with oil. However, oil, such as in a motor, breaks down under hot conditions; thus, the known phrase “time to get an oil change.” There is a need for a compound that lubricates nonhuman mechanical interfaces with the ability to remain structurally sound under higher temperatures than conventional oil and other lubricating substances.

### SUMMARY OF INVENTION

Carbon is one of the most commonly used natural elements in our world in carbon. It is extremely plentiful and inexpensive. In 1996, the Nobel Prize in Chemistry was awarded to several scientists who discovered that carbon existed in more than only two forms graphite and diamonds. They discovered that a third molecular form or allotrope of carbon exists, namely fullerenes. Shaped much like a soccer ball, a fullerene is a combination of carbon atoms arranged together as interlocked pentagons and hexagons. A fullerene is also known as C60.

Upon fullerenes discovery, many scientists were excited to see if fullerenes could be applied to the fields of super-conductivity, HIV protease inhibitors, and carbon nanotubes and nanowires. Unfortunately, there have not been many commercially viable fullerene applications.

The present invention employs fullerenes as a lubricant in human joints, and other nonhuman mechanical interfaces.

### DETAILED DESCRIPTION

Administering fullerenes for use in lubrication of joints is the present invention. The synovial fluid and cartilage that allow a joint to move and flex need to be replaced if the fluid and cartilage is lost. Else, the joint will cause pain and discomfort, and cease to function properly. The present invention asserts that fullerenes are to be used so that lubrication can be restored.

Moreover, the present invention administers fullerenes in the lubrication of mechanical joints in motors and other non-living interfaces. Fullerenes have a relatively high tolerance for temperature 1500 degrees Fahrenheit. Thus, will not breakdown as do typical types of motor oil. Even in situations where heat is not a factor, fullerenes are inter, and thus, can remain functional as the perfect lubricant so long as there is no loss of fullerenes from a space. If loss occurs, then more fullerenes can simply be applied to the interface to be lubricated.

Rather than attempt to repair a joint″s lack of naturally or biologically occurring fluid and cartilage, the present invention is a crutch or artificial replacement for the fluid and cartilage. If synovial fluid and cartilage remains in a joint, then the present invention merely supplements the amount of fluid and cartilage that remains in the joint. The user would be given an amount of fullerenes that restores desired movement and flex to a joint. If synovial fluid and cartilage is essentially lacking in a joint, then the present invention completely takes the place of the cartilage and fluid to allow the bones at the joint to move relative to one another without abrasion.

The present invention acts as a lubricant much the way soccer balls roll over one another if placed in a bin. In other words, the fullerenes do not react with one another, and they are geometrically shaped to roll over one another and not become interlocked. As pressure is applied to fullerenes, they roll over one another so that they act as if they were cartilage and synovial fluid, or as if they were conventional oil.

As per the present invention, the fullerenes are to be administered directly into the joint space using a syringe and needle. No other method of administration could be expected to result in adequate concentrations of fullerenes to produce the desired effect; that is, delivery to the joint space could not be accomplished by injection at a remote or adjacent site nor by the bloodstream.

In the preferred embodiment of the present invention, a 5 mg is effective to provide billions of fullerenes in the joint space. The fullerene concentration in the joint space must be maintained; and thus, depending upon the location of the joint and the level of joint activity of the user, the fullerenes will need to be reintroduced into the joint space over time as initially introduced. Because variation in joint space size in different human beings is very slight, it is not a consideration of any importance. In an alternative embodiment of the present invention, hyaluronic acid can be introduced into the joint space to delay or prevent diffusion of fullerenes from the joint space.

In its molecular form, carbon is inert and does not produce inflammatory or immune body responses. Fullerenes are even more compatible with the human body because their unique spherical form is very stable. The kidneys of the human body naturally remove the fullerenes from the bloodstream, and the fullerenes are then made part of common excretions in their unchanged form the form that they were in upon introduction to the human body and the form that they were in throughout their path in the human body. Fullerenes have no adverse effects on the kidneys. In addition, fullerenes can be eliminated from the human body by the liver into the intestine. Because of their minute atomic size, fullerenes pose no hazard as blood vessel blockages. Further, because of their ability to roll over one another, fullerenes do not stack in such a way that a blockage could occur. Insoluble in bodily fluids, fullerenes cannot pass into bodily tissues or dissolve in the blood. Thus, because fullerenes are not soluble, they naturally tend to stay localized to the joint space wherein they are introduced. While diffusion can occur, they would be removed from the human body as aforementioned.

Fullerenes are natural electron acceptors, and thus, have antioxidant and anti-inflammatory activity, promoting healing of bone and cartilage damage typical at faulty joints. Thus, while drugs could be introduced with the fullerenes, there is little reason for it to occur unless immediate pain relief is sought and in such case, common pain relievers are contemplated as being part of the administration.

The upper limits of fullerene dosage is not a huge issue because of fullerene″s non reactivity and relatively small dose being administered. There is no “ramping up” of dosage necessary for fullerenes because the dose needed to be effective should be administered on a regular basis, as needed. There is no need for “ramping down” of dosage necessary when removing the user from fullerene therapy because fullerenes are not pharmaceutical, and thus, there is no need for slow withdrawal.

Temperature of fullerenes is a non issue, as the dosage of fullerenes being administered is small, and the human body will quickly warm or cool the fullerenes introduced into the joint space.

The present invention, as aforementioned, sets forth the use of fullerenes, in a similar fashion, to nonhuman mechanical interfaces that require lubrication. Whereas oil breaks down, oftentimes at 650 degrees Fahrenheit, fullerenes do not. Thus, even conventional oil can be mixed with fullerenes to provide greater tolerance and less undesirable chemical breakdown residue, while still fully enabling lubrication.

The present invention is not solely limited to the embodiments described above, but also is considered to be any and all embodiments within the scope of the following claims.

1. The method of treating a joint, comprising: injecting approximately 5 mg of fullerenes into a joint space.

2. The method of claim 1, further comprising injecting more fullerenes into the joint space periodically.

3. The method of treating an interface in need of lubrication, comprising:
filling an interface with fullerenes.

4. The method of claim 3, further comprising mixing oil with said fullerenes.
